(19) United States
(12) Patent Application Publication
(10) Pub. No.: US 2021/0327567 A1
(43) Pub. Date: Oct. 21, 2021

(54) Machine-Learning Based Surgical Instrument Recognition System and Method to Trigger Events in Operating Room Workflows

(71) Applicant: Explorer Surgical Corp., Chicago, IL (US)

(72) Inventors: Eugene Aaron FINE, Northbrook, IL (US); Jennifer Porter FRIED, Chicago, IL (US)

(21) Appl. No.: 17/232,193

(22) Filed: Apr. 16, 2021

Related U.S. Application Data
(60) Provisional application No. 63/012,478, filed on Apr. 20, 2020.

(51) Int. Cl. G16H 40/20; G06K 9/00; G16H 70/20; G16H 40/67; G06N 3/08

(52) U.S. Cl. CPC G16H 40/20; G06K 9/00718; G06N 3/08; G16H 40/67; G16H 70/20

(57) ABSTRACT

Technologies are provided that define surgical team activities based on instrument-use events. The system receives a real-time video feed of the surgical instruments prep area and detects unique surgical instruments and/or materials entering/exiting the video feed. The detection of these instruments and/or materials trigger instrument use events that automatically advance the surgical procedure workflow and/or trigger data collection events.

### RELATED APPLICATIONS

This application claims the benefit of U.S. Provisional Application No. 63/012,478 filed Apr. 20, 2020 for “Machine-Learning Based Surgical Instrument Recognition System and Method to Trigger Events in Operating Room Workflows,” which is hereby incorporated by reference in its entirety.

### TECHNICAL FIELD

This disclosure relates generally to an automatic workflow management system that manages surgical team activities in an operating room based on instrument-use events. In particular, this disclosure relates to a machine-learning based system that detects objects entering/exiting the field of view of a video feed in the operating room to trigger instrument-use events that automatically advance the surgical procedure workflow and/or trigger data collection events and/or other events.

### BACKGROUND

Surgeries are inherently risky and expensive. Ultimately, the cost and success of a patient's surgical care are determined inside the operating room (OR). Broadly speaking, surgical outcomes and related healthcare costs are multifactorial and complex. However, several key variables (e.g., length of surgery, efficient use of surgical resources, and the presence/absence of peri-surgical complications) can be traced back to coordination and communication among the nurses, surgeons, technicians, and anesthesiologists that make up the surgical team inside the OR. Teamwork failures have been linked to intra- and post-operative complications, wasted time, wasted supplies, and reduced access to care. Indeed, more well-coordinated teams report fewer complications and lower costs. To enhance team coordination, the focus must be on optimizing and standardizing procedure-specific workflows and generating more accurate, granular data regarding what happens during surgery.

Across many other medically-related fields, technology has been used successfully to improve and streamline communication and coordination (e.g., electronic health records (EHRs), patient portals, engagement applications, etc.) with significant positive impacts on patient health and healthcare costs. In contrast, analogous technologies aimed at penetrating the “black box” of the OR have lagged behind. In many cases surgical teams still rely on analog tools—such as a preoperative “time out” or so-called preference cards—to guide coordination, even during surgery. Unsurprisingly, preferences and approaches vary widely among surgeons, comprising one of the main reasons why OR teams that are familiar with each other tend to be associated with better patient outcomes. However, relying on analog support and/or familiarity among teams is inefficient and unsustainable. Not only are analog-based tools inherently difficult to share and optimize, they are rarely consulted during the surgical case itself and they fail to address the many role-specific tasks or considerations that are critical to a successful procedure.

In addition, a lack of digital tools also contributes to the dearth of data on what actually goes on inside the OR. However, getting this technology into the OR is just the first step. Existing tools rely on manual interactions (application user interface) to advance the surgical workflow and trigger data collection. The manual interaction requirement is a barrier to routine use and, thus, limits the accuracy and completeness of the resulting datasets. Lapses in efficiency and OR team coordination lead to poorer patient outcomes, higher costs, etc.

There are over 150,000 deaths each year among post-surgical patients in the U.S., and post-operative complications are even more common. One of the best predictors of poor surgical patient outcomes is length of surgery. Relatedly, surgeries that run over their predicted time can have a domino effect on facilities, personnel, and resources, such that other procedures and, ultimately, patient health outcomes are negatively affected. Improvements in surgical workflows and individual case tracking are needed to address this problem. Every member of the OR team has a critical role in ensuring a good patient outcome, thus even minor mishaps can have significant consequences if it results in diverted attention or delays. Disruptions, or moments in a case at which the surgical procedure is halted due to a missing tool, failure to adequately anticipate or prepare for a task, or a gap in knowledge necessary to move onto the next step in a case, are astoundingly pervasive; one study finds nurses leave the operating table an average of 7.5 times per hour during a procedure, and another reports nurses are absent an average of 16% of the total surgery time.

Minor problems are exacerbated by a lack of communication or coordination among members of the surgical team. Indeed, prior research estimates as much as 72% of errors in the OR are a result of poor team communication and coordination, a lack of experience, or a lack of awareness among OR personnel. One strategy to minimize such errors is to develop highly coordinated teams who are accustomed to working together. Indeed, targeted strategies to improve coordination and communication are effective in the OR setting. However, it is unrealistic to expect that every surgery can be attended by such a team. Another strategy is to implement a standardized workflow delivered to the team at the point-of-care.

There is also a lack of intraoperative data collection in the OR. The OR is a “data desert.” Physical restrictions to the space and the need to minimize any potential sources of additional clutter, distraction, or burden to the surgical team, whether physical or mental, have made the OR a particularly difficult healthcare setting to study. As a result the literature surrounding OR best practices and data-driven interventions to improve efficiency and coordination is notably thin. The data that are available, including standard administrative data (e.g., “start” and “stop” times, postoperative outcomes), tool lists, and post-hoc reports from the surgeon or other members of the OR team, are insufficient to understand the full spectrum of perioperative factors impacting patient outcomes. Perhaps more significant, these data lack the necessary precision and granularity with which to develop anticipatory guidance for optimizing patient care and/or hospital resources.

Unpredictable “OR times” (total time from room-in to room-out) is a common problem that can throw off surgical schedules, resulting in canceled cases, equipment conflict, and the need for after-hours procedures, all of which can translate to unnecessary risks for patients and avoidable hospital expenses. After-hours surgeries are particularly problematic, as they typically involve teams unused to working together, more limited access to ancillary services, such as radiology or pathology, and are associated with a higher rate of complications and costs. Relying on physician best guesses and/or historical OR time data is not sufficient. Moreover, past efforts to generate more accurate prediction models using the coarse data available still fall short. Advancing towards more accurate and more fine-grained data are critical to improve this aspect of surgical care. In addition, at the individual patient level, the ability to track specific events or observations during surgery in real-time (for example, a cardiac arrest event during surgery or evidence of wound infection) has the potential to improve post-operative care (intense cardiac monitoring or a stronger course of antibiotics).

### SUMMARY

According to one aspect, this disclosure provides a computing device for managing operating room workflow events. The computing device includes an instrument use event manager to: (i) define a plurality of steps of an operating room workflow for a medical procedure; and (ii) link one or more instrument use events to at least a portion of the plurality of steps in the operating room workflow. There is an instrument device recognition engine to trigger an instrument use event based an identification and classification of at least one object within a field of view of a real-time video feed in an operating room (OR). The system also includes a workflow advancement manager to, in response to the triggering of the instrument use event, automatically: (1) advance the operating room workflow to a step linked to the instrument use event triggered by the instrument device recognition engine; and/or (2) perform a data collection event linked to the instrument use event triggered by the instrument device recognition engine.

According to another aspect, this disclosure provides one or more non-transitory, computer-readable storage media comprising a plurality of instructions stored thereon that, in response to being executed, cause a computing device to: define a plurality of steps of an operating room workflow for a medical procedure; link one or more instrument use events to at least a portion of the plurality of steps in the operating room workflow; trigger an instrument use event based an identification and classification of at least one object within a field of view of a real-time video feed in an operating room (OR); and automatically, in response to triggering the instrument use event, (1) advance the operating room workflow to a step linked to the instrument use event triggered by the instrument device recognition engine; and/or (2) perform a data collection event linked to the instrument use event triggered by the instrument device recognition engine.

According to a further aspect, this disclosure provides a method for managing operating room workflow events. The method includes the step of receiving a real-time video feed of one or more of instrument trays and/or preparation stations in an operating room, which is broadly intended to mean any designated viewing area identified as suitable for collecting instrument use events. One or more surgical instrument-use events are identified based on a machine learning model. The method also includes automatically advancing a surgical procedure workflow and/or triggering data collection events as a function of the one or more surgical instrument-use events identified by the machine learning model.

### BRIEF DESCRIPTION OF THE DRAWINGS

The concepts described herein are illustrated by way of example and not by way of limitation in the accompanying figures. For simplicity and clarity of illustration, elements illustrated in the figures are not necessarily drawn to scale. Where considered appropriate, reference labels have been repeated among the figures to indicate corresponding or analogous elements.

FIG. 1 is a simplified block diagram of at least one embodiment of an automatic workflow management system;

FIG. 2 is a simplified block diagram of at least one embodiment of various environments of the system of FIG. 1;

FIG. 3 is a simplified flow diagram of at least one embodiment of a method for automatically advancing a surgical workflow;

FIG. 4 is a top view of a tray showing a plurality of instruments for which a machine learning model could be trained to detect instruments according to at least one embodiment of this disclosure;

FIG. 5 illustrates a plurality of photographs of an instrument at varying orientations that can be used to train the machine learning model to recognize the instruments according to at least one embodiment of this disclosure;

FIGS. 6-7 illustrate an example video feed in an operating room showing the machine learning model recognized various instruments according to at least one embodiment of this disclosure;

FIG. 8 illustrates a confusion matrix resulting from object recognition of the initial testing set of 20 surgical instruments in which predictions are represented by rows and object identifiers are presented in columns according to at least one embodiment of this disclosure;

FIG. 9 illustrates improvements in loss during training a machine learning model to detect a plurality of instruments according to at least one embodiment of this disclosure;

FIG. 10 illustrates improvements in accuracy during training a machine learning model to detect a plurality of instruments according to at least one embodiment of this disclosure; and

FIG. 11 is a simplified flow diagram of at least one embodiment of a method for defining workflows linked with instrument use events.

### DETAILED DESCRIPTION OF THE DRAWINGS

While the concepts of the present disclosure are susceptible to various modifications and alternative forms, specific embodiments thereof have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one A, B, and C” can mean (A); (B); (C); (A and B); (A and C); (B and C); or (A, B, and C). Similarly, items listed in the form of “at least one of A, B, or C” can mean (A); (B); (C); (A and B); (A and C); (B and C); or (A, B, and C).

The disclosed embodiments may be implemented, in some cases, in hardware, firmware, software, or any combination thereof. The disclosed embodiments may also be implemented as instructions carried by or stored on a transitory or non-transitory machine-readable (e.g., computer-readable) storage medium, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

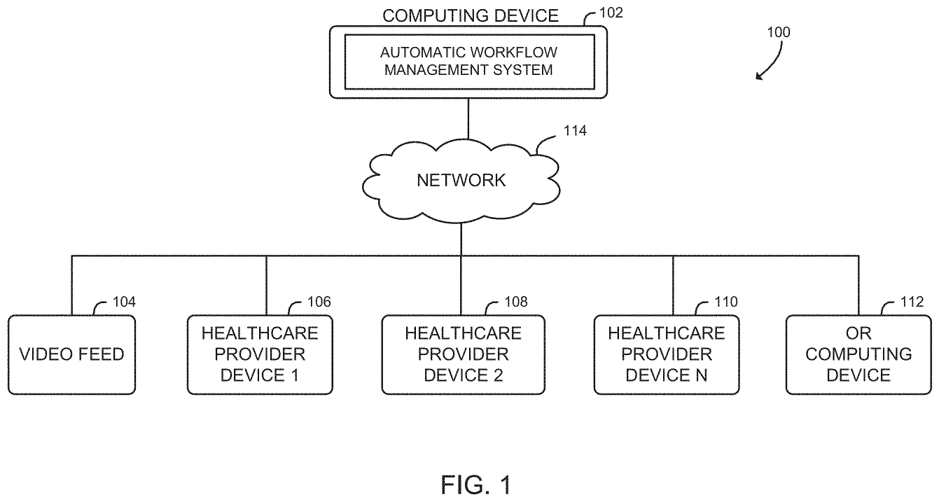

Referring now to FIG. 1, there is shown an embodiment of a system 100 for automatic workflow management that supports an entire operating room (OR) team to fully automate workflow support software by automatically advancing steps in the workflow based on an analysis of a real-time video feed showing a surgery in the OR. In some embodiments, the system 100 could be integrated with the ExplORer Live™ software platform by Explorer Surgical Corp. of Chicago, Ill. Instead of manually advancing to the next step in the workflow as with the existing version of ExplORer Live™, however, the system 100 automatically advances to the next step in the workflow and/or performs data collection based on detection of instrument use events. For example, the system 100 could leverage machine learning and artificial intelligence (ML/AI) to automatically link instrument and/or material use with steps in the surgery workflow. In some embodiments, the system 100 intelligently recognizes which step of the surgical procedure the OR team is currently performing. For example, the system 100 may identify specific surgical instrument-use events that can be accurately identified using AI/ML object recognition technologies, and link surgical instrument-use events to surgical procedure workflow. The term “instrument use events” is broadly intended to mean any instrument, tool, material and/or other object identified within the OR that may be linked to a surgical procedure workflow and is not intended to be limited to identification of instruments.

In some cases, for example, the system 100 may advance steps in the workflow and/or trigger data collection based on a machine learning engine that automatically recognizes the presence and/or absence of instruments and/or materials in the OR from the real-time video feed. The terms “surgery” and “medical procedure” are broadly intended to be interpreted as any procedure, treatment or other process performed in an OR, treatment room, procedure room, etc. The term “operating room” or “OR” is also broadly intended to be interpreted as any space in which medical treatments, examinations, procedures, etc. are performed. Moreover, although this disclosure was initially designed for use in a clinical setting (i.e., the OR), embodiments of this disclosure have applicability as a teaching/training tool. For example, nursing staff can use the material to fast-track “onboarding” of new nurses (a process that can take six months or longer in some cases); educators can use material to train medical students or residents before they enter the OR; and physicians can review modules developed by their colleagues to learn about alternative surgical approaches or methods. Accordingly, the term “OR” as used herein is also intended to include such training environments.

In some cases, the system 100 automates data collection within the OR. In some embodiments, for example, the system 100 provides time-stamped automatic data collection triggered by recognizing the presence and/or absence of certain instruments and/or materials in the OR from the real-time video feed. This data collected automatically in the OR may lead to insights into how events during surgery may predict post-operative outcomes, and by fully automating data collection, embodiments of the system 100 will increase accuracy of such data. In addition, as the recent COVID-19 pandemic has dramatically illustrated, it is beneficial to minimize the total number of personnel required for a given procedure, both to reduce potential hazards and optimize efficient use of personal protective equipment (PPE), making automated data collection highly advantageous.

In the embodiment shown, the system 100 includes a computing device 102 that performs automatic workflow management in communication with one or more computing devices 104, 106, 108, 110, 112 in the OR over a network 114. For example, the computing device 104 may be one or more video cameras that stream real-time video data of a field of view in the OR to the computing device 102 over the network 114. The computing devices 106, 108, 110 could be computing devices used by one or more members of the OR team to display the steps in the workflow and/or other information specific to that stage in the surgery. For example, in some cases, at least a portion of the OR team could each have their own computing device with a role-based workflow individualized for that particular member of the OR team. Depending on the circumstances, an OR may include a computing device 112 that is shared by multiple members of the team.

For example, the appropriate step in a surgery workflow could be determined by the computing device 102 based on analysis of the video feed 104, and communicated to one or more of the other computing devices 106, 108, 110, 112 to display the appropriate step. In some embodiments, the appropriate step could be role-based for each computing device 106, 108, 110, 112, and therefore each device 106, 108, 110, 112 may display a different step depending on the user's role.

Consider an example in which computing device 106 and computing device 108 are being used by different users in the OR with different roles mapped to different steps in the surgery workflow. When the computing device 102 recognizes Instrument 1 being used based on the video feed, the computing device 102 may instruct computing device 106 to advance to Step B, which results in computing device 106 displaying Step B; at the same time, computing device 102 may instruct computing device 108 to advance to Step Y, which results in computing device 108 to display Step Y. Upon the computing device 102 recognizing Instrument 1 being put away, the computing device 102 may instruct computing device 106 to display Step C and computing device 108 to display Step Z. In this manner, the presence and/or absence of certain instruments, tools, and/or materials in the video feed 104 may trigger the computing device 102 to communicate certain events to other computing devices.

In some embodiments, as explained herein, the computing device 102 may include a machine learning engine that recognizes the presence and/or absence of certain instruments and/or materials in the OR, which can be triggered for advancing the workflow and/or data collection. Depending on the circumstances, the computing device 102 could be remote from the OR, such as a cloud-based platform that receives real-time video data from one or more video cameras in the OR via the network 114 and from which one or more functions of the automatic workflow management are accessible to the computing devices 106, 108, 110, 112 through the network 114. In some embodiments, the computing device 102 could reside within the OR with one or more onboard video cameras, thereby alleviating the need for sending video data over the network 114. Although a single computing device 102 is shown in FIG. 1 for purposes of example, one skilled in the art should appreciate that more than one computing device 102 could be used depending on the circumstances. Likewise, although FIG. 1 illustrates a plurality of computing devices 104, 106, 108, 110, 112 that are capable of accessing one or more functions of the computing device 102 over the network 114, a single computing device could be provided depending on the circumstances. Additionally, although a single video feed 104 is shown in FIG. 1, there could be multiple cameras with different camera angles feeding video from the OR depending on the circumstances.

The computing devices 102, 104, 106, 108, 110, 112 may be embodied as any type of computation or computer device capable of performing the functions described herein, including, without limitation, a computer, a server, a workstation, a desktop computer, a laptop computer, a notebook computer, a tablet computer, a mobile computing device, a wearable computing device, a network appliance, a web appliance, a distributed computing system, a processor-based system, and/or a consumer electronic device. Additionally or alternatively, the computing device 102 may be embodied as a one or more compute sleds, memory sleds, or other racks, sleds, computing chassis, or other components of a physically disaggregated computing device. Depending on the circumstances, the computing device 102 could include a processor, an input/output subsystem, a memory, a data storage device, and/or other components and devices commonly found in a server or similar computing device. Of course, the computing device 102 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory, or portions thereof, may be incorporated in the processor in some embodiments.

The computing devices 102, 104, 106, 108, 110, 112 include a communication subsystem, which may be embodied as any communication circuit, device, or collection thereof, capable of enabling communications between the computing device 102, video feed 104 and other computing devices 106, 108, 110, 112 over the computer network 114. For example, the communication subsystem may be embodied as or otherwise include a network interface controller (NIC) or other network controller for sending and/or receiving network data with remote devices. The NIC may be embodied as any network interface card, network adapter, host fabric interface, network coprocessor, or other component that connects the computing device 102 and computing devices 104, 106, 108, 110, 112 to the network 106. The communication subsystem may be configured to use any one or more communication technology (e.g., wired or wireless communications) and associated protocols (e.g., Ethernet, InfiniBand®, Bluetooth®, Wi-Fi®, WiMAX, 3G, 4G LTE, 5G, etc.) to effect such communication.

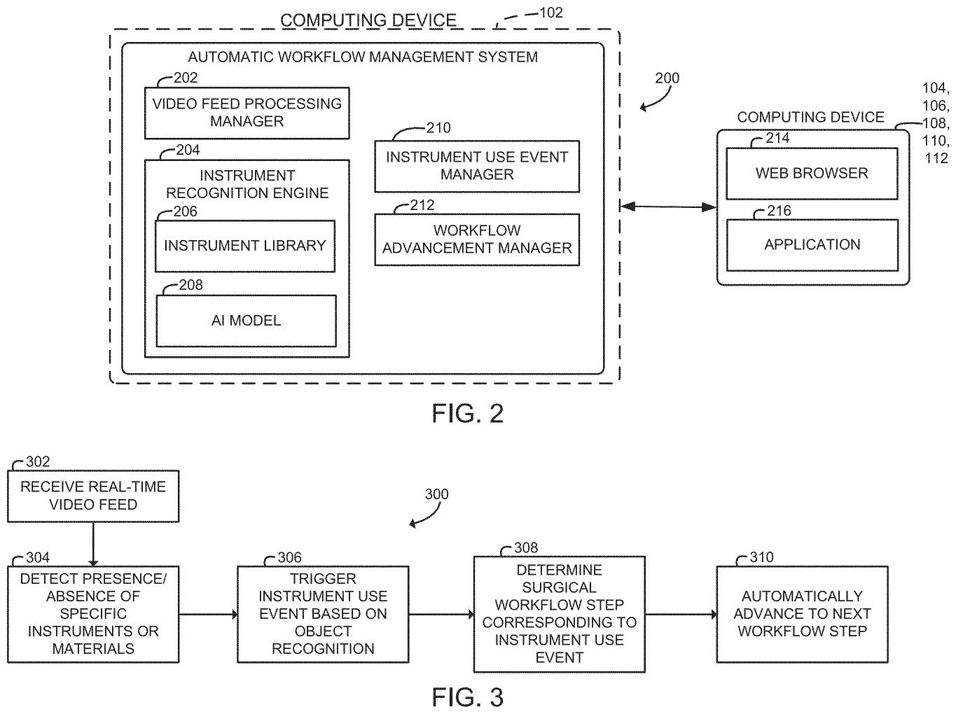

The computing devices 106, 108, 110, 112 are configured to access one or more features of the computing device 102 over the network 114. For example, the computing device 102 may include a web-based interface or portal through which users of the computing devices 106, 108, 110, 112 can interact with features of the computing device 102 using a browser, such as Chrome™ by Google, Inc. of Mountain View, Calif. (see browser 214 on FIG. 2). Embodiments are also contemplated in which the computing devices 106, 108, 110, 112 may be mobile devices running the Android™ operating system by Google, Inc. of Mountain View, Calif. and/or mobile devices running iOS™ operating system by Apple Inc. of Cupertino, Calif. on which software has been installed to perform one or more actions according to an embodiment of the present disclosure. For example, the computing devices 104 may have an app installed that allows a user to perform one or more actions described herein (see app 216 on FIG. 2). In some embodiments, the computing devices 106, 108, 110, 112 may be a laptop, tablet, and/or desktop computer running the Windows® operating system by Microsoft Corporation of Redmond, Washington on which software, such as app 216, has been installed to perform one or more actions. Although the system 100 is described as being a cloud-based platform accessible by the remote computing devices 104, 106, 108, 110, 112 in some embodiments one or more features of the server 102 could be performed locally on the remote computing devices 104.

Referring now to FIG. 2, in an illustrative embodiment, the computing device 102 establishes an environment 200 during operation for an automatic workflow management system. The illustrative environment 200 includes a video feed processing manager 202, an instrument recognition engine 204 with an instrument library 206 and AI model 208, an instrument use event manager 210, and a workflow advancement manager 212. As shown, the various components of the environment 200 may be embodied as hardware, firmware, software, or a combination thereof. As such, in some embodiments, one or more of the components of the environment 200 may be embodied as circuitry or collection of electrical devices (e.g., video feed processing manager circuitry, instrument recognition engine circuitry, instrument use event manager circuitry, and workflow advancement manager circuitry). Additionally or alternatively, in some embodiments, those components may be embodied as hardware, firmware, or other resources of the computing device 102. Additionally, in some embodiments, one or more of the illustrative components may form a portion of another component and/or one or more of the illustrative components may be independent of one another.

The video feed processing manager 202 is configured to receive a real-time video from one or more cameras in the OR. For example, the video feed processing manager 202 could be configured to receive video data communications from one or more cameras in the OR via the network 114. As discussed above, the video data provides a field of view in the OR for analysis by the instrument recognition engine 204 to determine triggers for workflow advancement and/or data collection. In some cases, the video feed processing manager 202 could be configured to store the video data in memory or a storage device for access by the instrument recognition engine 204 to analyze the video substantially in real time.

The instrument recognition engine 204 is configured to recognize instruments, tools, materials, and/or other objects in the OR using AI/ML. For example, the instrument recognition engine 204 may go from object images to accurate object detection and classification using innovative AI/ML deep learning techniques. To accomplish this, in some embodiments, the instrument recognition engine 204 includes a convolutional neural network (CNN). A CNN “recognizes” objects by iteratively pulling out features of an object that link it to increasingly finer classification levels.

In some cases, the RetinaNet algorithm may be used for object detection and classification. RetinaNet is a highly accurate, one-stage object detector and classifier. It is the current leading approach in the field (used in self-driving car technology, among other applications), boasting significant improvements in accuracy over other techniques. Briefly, RetinaNet is a layered algorithm comprising two key sub-algorithms: a Feature Pyramid Network (FPN), which makes use of the inherent multi-scale pyramidal hierarchy of deep CNNs to create feature pyramids and a Focal Loss algorithm, which improves upon cross-entropy loss to help reduce the relative loss for well-classified examples by putting more focus on hard, misclassified examples. This in turn makes it possible to train highly accurate dense object detectors in the presence of vast numbers of easy background examples.

A prototype object recognition model of the instrument recognition engine 204 that the inventors developed during Phase 1-equivalent work achieved 81% accuracy in identifying an initial set of 20 surgical instruments. As discussed herein, there are key technical innovations to improve accuracy by addressing complex environmental conditions that may present within the OR. Specifically, to the RetinaNet-based model, embodiments of this disclosure layer tools that address the following special cases:

Blur and specular reflection: During a surgical procedure, objects may become difficult to detect due to blur and/or changes in specular reflection. In some embodiments, the instrument recognition engine 204 applies a combination of algorithms to combat these issues, including the Concurrent Segmentation and Localization for Tracking of Surgical Instruments algorithm, which takes advantage of the interdependency between localization and segmentation of the surgical tool.

Unpredictable object occlusion: During a procedure, instruments may become occluded on the tray or stand, which then hinders neural networks' ability to detect and classify objects. Embodiments of this disclosure includes the Occlusion Reasoning for Object Detection algorithm, which can handle spatially extended and temporally long object occlusions to identify and classify multiple objects in the field of view.

The inventors have completed Phase 1-equivalent, proof-of-concept work of the instrument recognition engine 204 to show that a convolutional neural network (CNN) model can be built and trained to detect instrument-use events. In Phase 2, the library of instruments recognized by the model may be expanded to accommodate a wide range of surgical procedures, optimize the model to deal with complex images and use-case scenarios, and finally integrate the model within the software platform for beta testing in the OR.

Phase 1-Equivalent Work

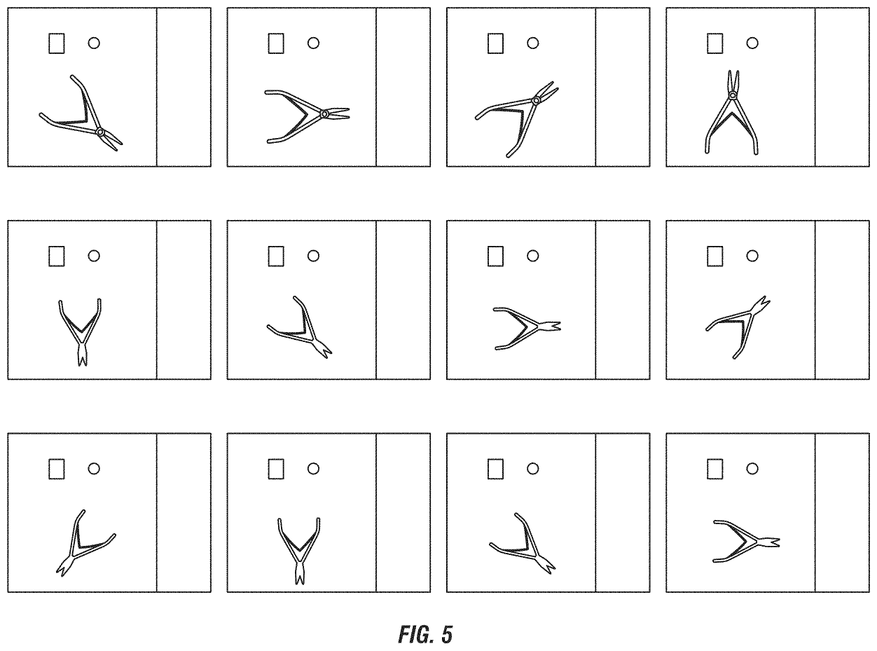

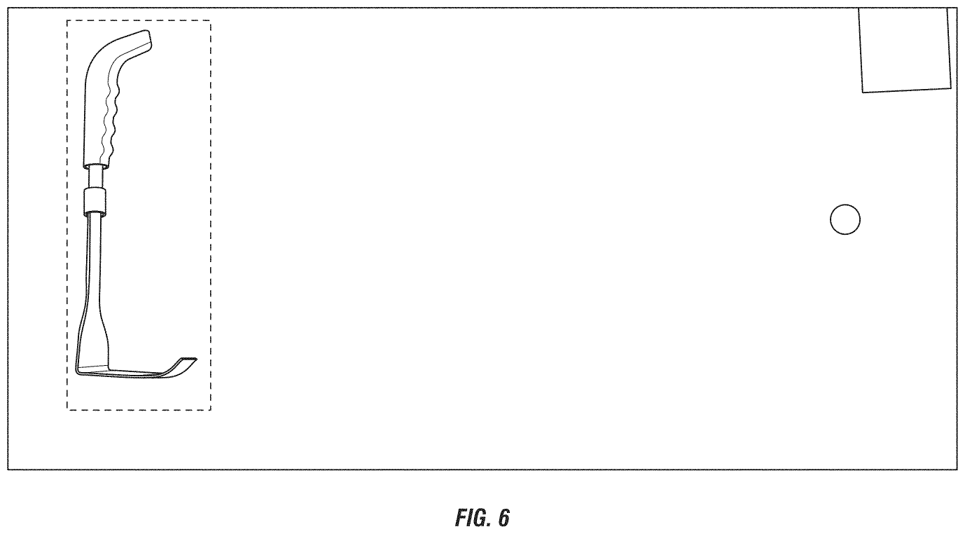

In developing the instrument recognition engine 204, Phase 1-equivalent work was performed to demonstrate the potential to detect instrument use events from real-time video feeds of instrument trays on moveable carts (i.e., mayo stands), mimicking the OR environment. For this phase of the project, a small-scale version of the instrument recognition engine 204 was built. A set of 20 commonly-used surgical instruments were used for this phase. (See FIG. 4). This initial set of instruments was designed to include both: (1) tools with similar but not identical form factors; and (2) tools with identical form factors but variations in size. Each instrument was imaged in various orientations (25° rotational shift between images; varying degrees of opened/closed, as applicable; ˜20 images per instrument) (See FIG. 5). Within each image, a bounding box around the instrument was defined, and labeled each image.

Once compiled, an artificial intelligence (AI) algorithm was developed for the instrument recognition engine 204 that could: (1) recognize and identify specific instruments; and (2) define instrument-use events based on when objects enter or leave the camera's field of view. In the field of AI/ML development, recent efforts to improve object recognition techniques have focused on (1) increasing the size of the network (now on the order of tens of millions of parameters) to maximize information capture from the image; (2) increasing accuracy through better generalization and the ability to extract signal from noise; and (3) enhancing performance in the face of smaller datasets. RetinaNet is the algorithm used to develop the initial prototype model, which is a single, unified network comprising one backbone network and two task-specific subnetworks. The backbone network computes a convolutional feature map of the entire image; of the two subnetworks: a Focal Loss algorithm limits cross-entropy loss (i.e., improves accuracy) by classifying the output of the backbone network; and a Feature Pyramid Network (FPN) performs convolution bounding box regression. Although development of the instrument recognition engine 204 is described with respect to RetinaNet, this disclosure is not limited to that specific implementation.

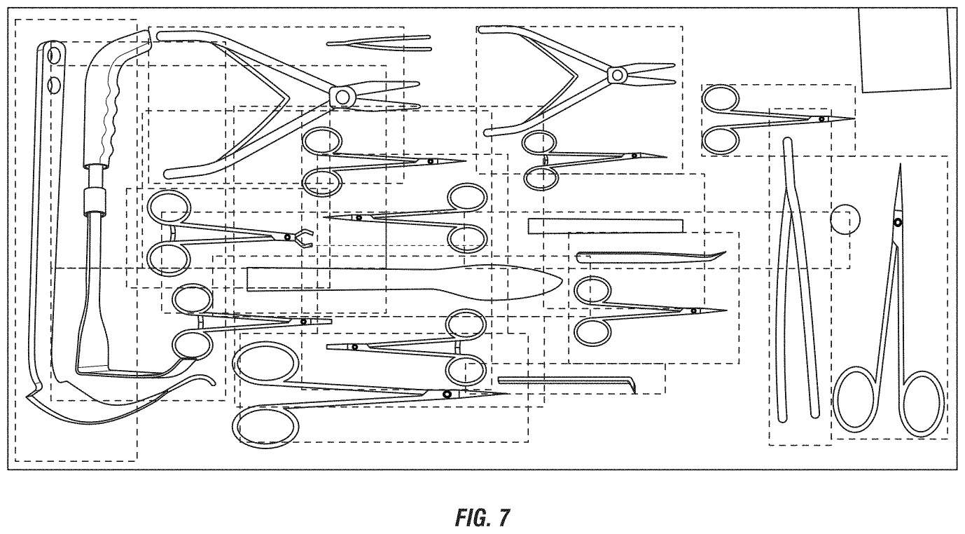

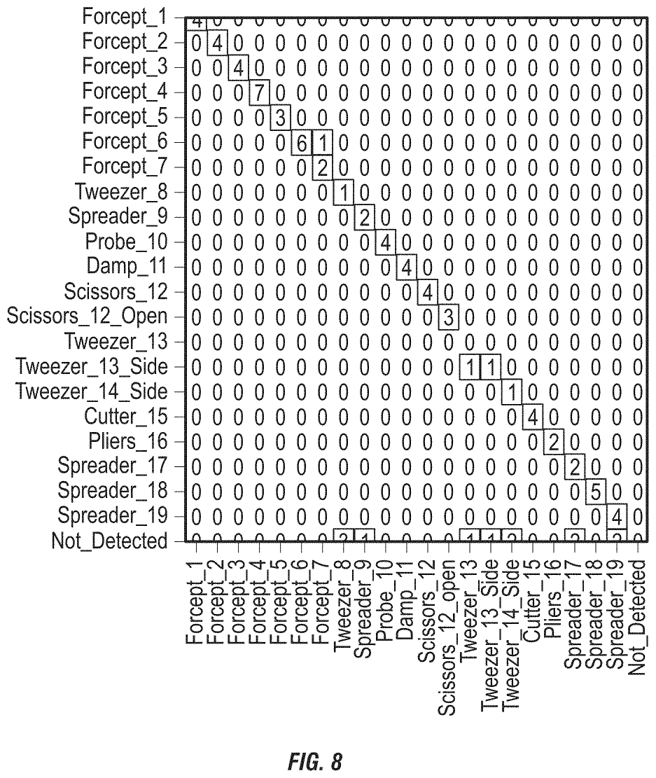

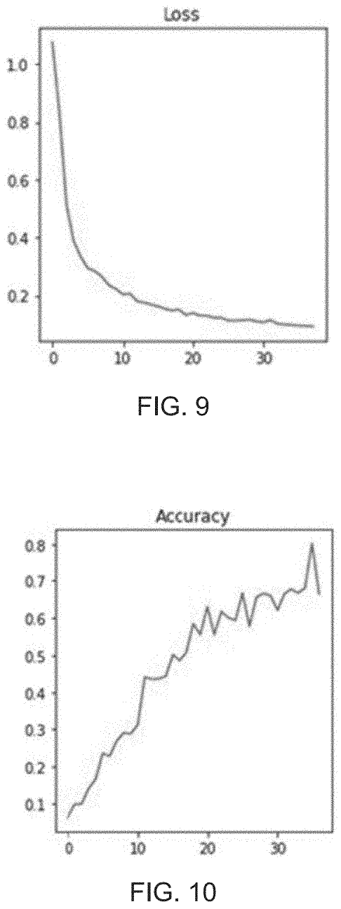

The instrument recognition engine 204 was then trained by using an instrument preparation station typical of most ORs (i.e., a mayo stand) and then a video camera was mounted from above to capture the entire set of instruments in a single view. For model testing, investigators dressed in surgical scrubs and personal protective equipment (PPE) proceeded to grab and replace instruments as if using them during a surgical procedure. The RetinaNet algorithm was applied to the live video feed, detecting instruments (identified by the bounding box) and classifying instruments by identification numbers for each instrument present within the field of view (See FIGS. 6-7). In the example shown in FIG. 6, there is shown a live-feed video in which the instrument recognition engine 204 detected, identified, and added bounding boxes to a surgical instrument added to a tray. In the example shown in FIG. 7, there is shown a live-feed video in which the instrument recognition engine 204 detected, identified, and added bounding boxes to a plurality of a surgical instruments added to a tray simulating a realistic scenario in the OR. Any time an instrument enters or exits the field of view, the instrument recognition engine 204 records this as an “instrument use event.” FIG. 8 illustrates a confusion matrix resulting from object recognition of the initial set of 20 surgical instruments in which predictions are represented by rows and object identifiers are presented in columns, which validated that the model identified the correct instrument use event 81% of the time. The persistent errors are related to more complex image scenarios, for instance, object occlusion or highly reflective surfaces, which are addressed herein. FIGS. 9 and 10 illustrate improvements in loss and accuracy, respectively, over the course of 40 epochs (x-axis) running the model. Based on these results, it is clear that applying ML/AI techniques in the instrument recognition engine 204 to recognize surgical instrument use events and then trigger surgical workflow advancement will work on a larger scale by expanding the set of instruments/materials recognized by the instrument recognition engine 204 and defining robust instrument use event-based triggers for workflows. Additionally, this testing identified two main sources of error in the instrument recognition engine 204: occlusion and reflected light. Embodiments of the instrument recognition engine 204 to address these conditions discussed herein.

Phase 2-Equivalent Work

Building off of the success of the Phase 1 work, the AI/ML model for the instrument recognition engine 204 was optimized with the image recognition algorithms for complex image scenarios unique to the OR, and integrated within the existing ExplORer Live™ platform. One objective of Phase 2 was to deliver fully automated, role-specific workflow advancement within the context of an active OR. Having demonstrated the potential to use AI/ML to link image recognition with instrument-use events in Phase 1, the training dataset was expanded to include a much wider range of surgical instruments, the model was optimized to handle more complex visual scenarios that are likely to occur during a procedure, and key trigger events were defined that are associated with workflow steps to effectively integrate the algorithm within the ExplORer Live™ software platform. In some cases, the instrument library 206 of the instrument recognition engine 204 could include 5,000 unique instruments or more depending on the circumstances and the AI model 208 is configured to accurately detect each of the unique instruments in the instrument library 206.

In Phase 1, >80% object recognition accuracy was achieved among 20 different instruments using a library of 400 images. Obviously, there are thousands of unique surgical instruments, supplies, and materials used across all possible surgical procedures. With the long-term goal of widespread implementation and culture change among surgical departments nationwide, or perhaps even globally, the instrument recognition engine 204 may be configured with a much broader object recognition capacity. Beginning with an analysis of product databases from selected major manufacturers, a list of about 5000 instruments was construed in three types of surgeries: (1) general, (2) neuro; and (3) orthopedic and spine for purposes of testing; however, the instrument recognition engine 204 could be configured for any type of instrument, tool, and/or material that may be used in the OR. The objective is to generate a library of images of these instruments to serve as a training set for the AI/ML model. The general approach to building an image library that supports unique object recognition will be based on lessons learned from proof-of-concept work plus iterative feedback as we optimize the model.

An objective in Phase 2 in expanding the set of unique instruments recognized by the instrument recognition engine 204 to maximize the applicability of the system 100 across hospitals and departments. An issue important for workflow management is uniquely defining key steps in a particular procedure. For example, clamps are a common surgical tool, often used repeatedly throughout a given procedure. Thus, clamps are unlikely to be a key object defining specific progress through a surgical workflow. On the other hand, an oscillating saw is a relatively specialized instrument, used to open the chest during heart surgery. The instrument-use event defined by the oscillating saw exiting the video frame is thus likely to serve as a key workflow trigger. In choosing the set of instruments to include in the expanded training set of images, there were multiple goals: (1) generate a set that covers a large proportion of surgeries, to maximize implementation potential; and (2) prioritize those instruments most likely to be associated with key workflow advancement triggers. General, orthopedic/spine, and neuro surgeries account for roughly 35% of surgeries performed in US hospitals each year. Thus, Phase 2 started by collecting a set of all instruments and materials involved in surgeries of these types, such as open and minimally invasive general surgeries (e.g., laparoscopic cholecystectomies, laparoscopic appendectomies, laparoscopic bariatric surgeries, hernia repairs, etc.), orthopedic surgeries (e.g., total joint replacements, fractures, etc.), and select neurosurgeries. An exhaustive set of such objects will include those linked to key workflow advancement steps. This approach is particularly amenable to scaling as the system 100 matures: material lists for other types of surgeries can be added incrementally in the future to the instrument recognition engine 204 in response to client needs.

In some embodiments, the number of images needed for the instrument recognition engine 204 to successfully identify an object (and differentiate it from other similar objects) varies depending on how similar/different an object is from other objects, or how many different ways it may appear when placed on the stand. For example, some instruments, like scalpels, would never be found lying perpendicular to the stand surface (blade directly up or down), thus there is no need to include images of scalpels in these orientations. In other instances, important identifying features may only be apparent in certain orientations (e.g., scissors). In the proof-of-concept study performed by the inventors, it was found that an average of 20 images per object (roughly 25° rotation between images, variation in open/closed configurations, position/sides configuration, variations in lightning conditions, etc.) were needed to achieve sufficiently accurate object recognition; however, more or less images may be provided per object depending on the circumstances. Phase 2 of development started with the same approach to imaging this expanded set of instruments. In some embodiments, the images could be taken using digital cameras and stored as .png files; however, other file types and other imaging techniques could be used depending on the circumstances. After raw images are taken, they are preprocessed (augmentation, resizing, and normalization) and stored in instrument library 206.

In Phase 2, there is an initial set of about 100,000 images (20 images×5,000 unique instruments) used to train/test the AI model 208. Depending on the circumstances, more images may be needed, either overall or for particular instruments. For example, accuracy testing may reveal errors caused by overfitting, which can be addressed by increasing the size of the training set (i.e., more images per instrument). Alternatively, errors may be linked to one or a handful of specific instruments, revealing the need for particular image variations of those objects. Other types of errors, for example those associated with particular environmental conditions like object occlusion, blur, or excessive light reflection, will be addressed through model optimization techniques. Ultimately, the instrument library 206 will be considered sufficient once the AI model 208 as a whole can accurately drive automated workflow advancement and data collection.

In building the proof-of-concept CNN model for Phase 1, the current leading object recognition network algorithm, RetinaNet, was implemented. As described herein, RetinaNet is considered to be the most advanced algorithm for detecting and identifying objects. Using this technique “out-of-the-box” led to greater than 80% accuracy among the initial set of 20 instruments for testing in Phase 1. The remaining inaccuracies are most likely due to “edge cases” in which environmental complexity, such as lightning conditions, image blur, and/or object occlusion introduce uncertainty. One of the objectives of Phase 2 was to address these remaining sources of inaccuracies by layering in additional algorithms designed specifically for each type of scenario.

Starting with the RetinaNet model developed in the proof-of-concept phase (see Phase 1-equivalent work, above), the expanded training dataset was used to measure both overall accuracy and identify key sources of remaining inaccuracies. There appear to be three potential sources of inaccuracy:

Training set-dependent: Image dataset is insufficient to uniquely identify the target objects, resulting in overfitting errors.

Object-dependent: Some objects are more prone to image interference based on their shape and material. For example, relatively flat metallic objects may cause reflections, particularly within the context of the OR, that can obscure their shape or other key identifying features.

Environment-dependent: Activity inside the OR is often hectic and fast-paced. This increases the chances that images captured via live video stream are blurry, or that objects placed on the mayo stand become occluded from time to time by other objects or by the reaching arms of OR personnel.

These types of errors can be addressed using one or more of the following:

Training set-dependent errors. Errors caused by overfitting are relatively easy to identify (i.e., the model performs extremely well on the trained dataset, but is markedly less accurate when challenged with an untrained dataset). If overfitting is detected, the training dataset can be expanded to address this issue.

Object-dependent errors. To combat the issue of specular reflection, a Concurrent Segmentation and Localization algorithm can be implemented. This algorithm can be layered on top of the existing RetinaNet model to define segments of the target object so as to be able to extract key identifying information from visible segments even if other parts of the object are obscured, say because of a reflection. It is a technique that has received much attention recently, particularly in medical imaging applications.

Environment-dependent errors. Additional algorithm layers can be applied to handle instances of object blur or occlusion. The Concurrent Segmentation and Localization tool, mentioned above, is also useful in detecting objects amid blur. The Occlusion Reasoning algorithm uses the idea of object permanence to extrapolate key identifying features that may be blocked by something else.

Training-set dependent errors, which are likely to have a major impact on the AI model 208 accuracy, will be apparent early on and can be addressed through iterative use of the techniques described herein. Thus, in some embodiments, a RetinaNet-based model that includes both the Concurrent Segmentation and Localization and Occlusion Reasoning add-ons may be used. Model optimization will then proceed iteratively based on performance using simulated OR video feeds. Ultimately, not all of the objects in the dataset have the same strategic importance. For this reason, a range of accuracy thresholds may be acceptable for different instruments/materials depending on the circumstances. Accuracy thresholds could be established based on workflow event triggers. Once these event triggers are known, they can be linked to instrument-use events, thus revealing those instruments that are of the greatest strategic importance (i.e., require the highest tolerance point) as the model 208 is optimized for accuracy. Once the model 208 is trained and optimized on the expanded image set, the instrument recognition engine 204 could be integrated the existing ExplORer Live™ software platform. This will link the instrument recognition engine 204 to workflow advancement and data collection triggers, and then the instrument recognition engine 204 could be tested within a simulated OR environment.

Although the system 100 may include any number of workflows specific to unique medical procedures, some embodiments are contemplated in which workflow information for hundreds or thousands of procedure variations are provided. For the vast majority of these workflows, the instruments used will be covered by the expanded image set. Leveraging the workflow information for these procedure variations, key workflow advancement triggers, i.e., surgical events that are associated with the transition from one workflow step to the next can be identified. Once the trigger events are identified, linking instrument-use events to workflow advancement events can be coded.

One aspect of setting up the system 100 will be iterative feedback between efforts to identify workflow event triggers and optimizing the accuracy of the AI/ML model in identifying the instruments involved in those event triggers. Beginning with a circumscribed set of surgical procedures to be used, instrument use events will be identified that can be used to trigger advancement at each step of the procedure's workflow. Briefly, a scoring system may be defined that evaluates each instrument based on key metrics, including recognition accuracy, frequency of use during the procedure, and functional versatility. The score will then be used to identify instruments best suited to serve as “triggers”.

Once workflow event triggers are defined, an iterative approach can be adopted to optimize the model 208 to prioritize accuracy in identifying instrument-use events linked to each trigger. Thus, certain “edge case” scenarios may emerge as more important to address than others, depending on if there is an instrument-use event that invokes a given scenario. Testing/optimization could occur via (1) saved OR videos, which could be manually analyzed to measure model accuracy; and/or (2) use during live surgical procedures (observation notes could be used to determine the accuracy of workflow transitions detected by the model). Optimization will become increasingly targeted until the model 208 achieves extremely high accuracy in identifying any and all workflow advancement events. In some embodiments, the existing ExplORer Live™ software platform could be modified to trigger workflow advancement based on the output of the instrument recognition engine 204 rather than manual button-presses. For example, the instrument recognition engine 204, instrument library, and/or AI model 208 could be deployed on a dedicated server cluster in the AWS hosting environment and could interface with ExplORer Live™ via a RESTful API layer.

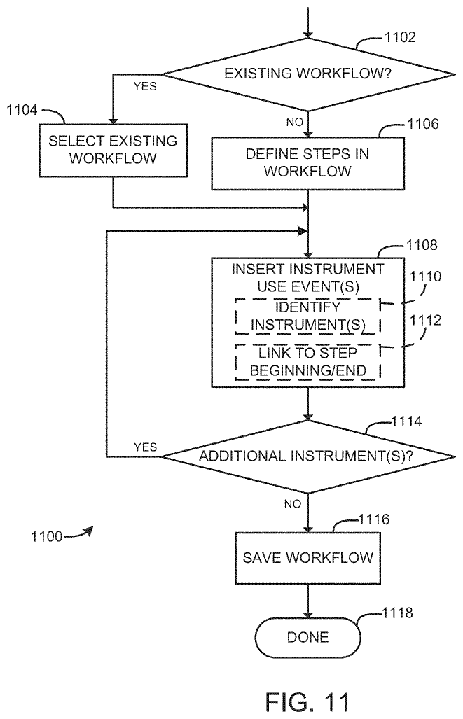

The instrument use event manager 210 is configured to define workflows with instrument use events. As discussed herein, surgical procedures may be defined by a series of steps in a workflow. In some cases, the workflow may be role-based in which each role may have individualized steps to follow in the workflow. For example, a nurse may have different steps to follow than a doctor in the workflow. In some embodiments, the instrument use event manager 210 may present an interface from which a user can define steps in a workflow and instrument use events. In some cases, the instrument use event manager 210 may open an existing workflow and add instrument use events. FIG. 11 illustrates a method 1100 for defining workflows with instrument use events that may be executed by the computing device 102. It should be appreciated that, in some embodiments, the operations of the method 1100 may be performed by one or more components of the environment 200 as shown in FIG. 2, such as the instrument use event manager 210. Also, it should be appreciated that the order of steps for the method 1100 shown in FIG. 11 are for purposes of example, and could be in a different order; likewise, depending on the circumstances, some steps shown in the method 1100 may be optional, or may not always be performed by the instrument use event manager 210. As illustrated, the method 1100 begins in block 1102 in which there is a determination whether there is an existing workflow to be selected or whether a new workflow is to be created. For example, the instrument use event manager 210 may include a user interface that allows the user to open an existing workflow for editing and/or create a new workflow. If the user wants to select an existing workflow, the method 1100 advances to block 1102 in which the user can select an existing workflow stored in storage. If the user wants to create a new workflow, the method 1100 moves to block 1106 from which the user can interact with an interface to define a plurality of steps in a workflow. Next, the method 1100 advances to block 1108 in which the user can identify instrument use events, such as an instrument entering/leaving the camera's field of view, which will trigger advancement in the workflow. For example, the identification of instrument use events could include an identification of a unique instrument 1110 that is linked to the beginning or end of a step in the workflow 1112. After the instrument use event is inserted into the workflow, a determination is made whether any additional instrument use events are desired to be added (Block 1114). If additional instrument use events are desired to be added, the method 1100 advances back to block 1108 until all instrument use events have been added to the workflow. Once no additional instrument use events are desired to be added, the method 1100 advances to block 1116 in which the workflow with linked instrument use events is saved, and then the method is done (block 1118).

The workflow advancement manager 212 is configured to manage advancement of steps in the workflow based on input received from the instrument recognition engine 204 indicating recognition of specific instruments and/or materials entering/leaving the field of view of the camera. As discussed herein, the workflows may be role specific, which means the step advancement could be different based on the role of the user and the instrument recognized by the instrument recognition engine 204. For example, the recognition of an oscillating saw by the instrument recognition engine 204 could cause the workflow advancement manager 212 to advance to Step X for a doctor role and Step Y for a nurse role in the workflow.

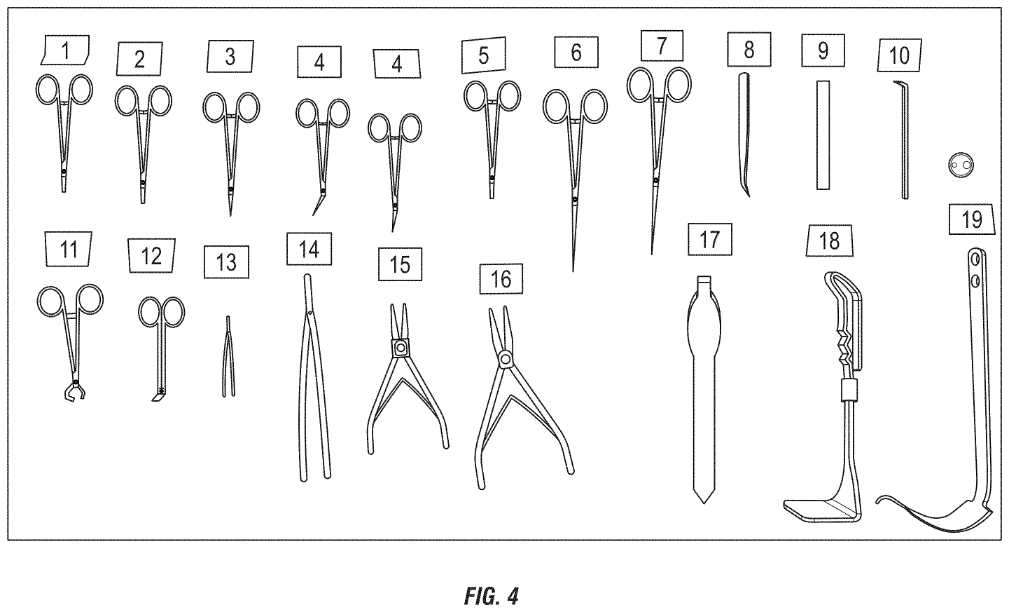

FIG. 3 illustrates operation of the system 100 according to some embodiments. As shown, the system 100 receives a real-time video feed of at least a portion of the OR (block 302). The instrument recognition engine 204 uses a ML/AI model 208 to determine the presence/absence of instruments and/or materials (block 304). Depending on one or more instrument-use events linked with a workflow, an instrument and/or material entering or leaving the field of view could trigger an instrument use event (block 306). The workflow advancement manager 212 determines the workflow step corresponding to the instrument use event and automatically advance to the appropriate step (blocks 308 and 310).

### EXAMPLES

Illustrative examples of the technologies disclosed herein are provided below. An embodiment of the technologies may include any one or more, and any combination of, the examples described below.

Example 1 is a computing device for managing operating room workflow events. The computing device includes an instrument use event manager to: (i) define a plurality of steps of an operating room workflow for a medical procedure; and (ii) link one or more instrument use events to at least a portion of the plurality of steps in the operating room workflow. There is an instrument device recognition engine to trigger an instrument use event based an identification and classification of at least one object within a field of view of a real-time video feed in an operating room (OR). The system also includes a workflow advancement manager to, in response to the triggering of the instrument use event, automatically: (1) advance the operating room workflow to a step linked to the instrument use event triggered by the instrument device recognition engine; and/or (2) perform a data collection event linked to the instrument use event triggered by the instrument device recognition engine.

Example 2 includes the subject matter of Example 1, and wherein: the instrument device recognition engine is configured to identify and classify at least one object within the field of view of the real-time video feed in the operating room (OR) based on a machine learning (ML) model.

Example 3 includes the subject matter of Examples 1-2, and wherein: the instrument device recognition engine includes a convolutional neural network (CNN) to identify and classify at least one object within the field of view of the real-time video feed in the operating room (OR).

Example 4 includes the subject matter of Examples 1-3, and wherein the instrument device recognition engine includes concurrent segmentation and localization for tracking of one or more objects within the field of view of the real-time video feed in the OR.

Example 5 includes the subject matter of Examples 1-4, and wherein: the instrument device recognition engine includes occlusion reasoning for object detection within the field of view of the real-time video feed in the OR.

Example 6 includes the subject matter of Examples 1-5, and wherein: the instrument device recognition engine is to trigger the instrument use event based on detecting at least one object entering the field of view of the real-time video feed in an operating room (OR).

Example 7 includes the subject matter of Examples 1-6, and wherein: the instrument device recognition engine is to trigger the instrument use event based on detecting at least one object leaving the field of view of the real-time video feed in an operating room (OR).

Example 8 includes the subject matter of Examples 1-7, and wherein: the workflow advancement manager is to determine the step linked to the instrument use event as a function of the identification and classification of the object detected by the instrument device recognition engine.

Example 9 includes the subject matter of Examples 1-8, and wherein: the workflow advancement manager is to determine the step linked to the instrument use event as a function of a role-based setting.

Example 10 is one or more non-transitory, computer-readable storage media comprising a plurality of instructions stored thereon that, in response to being executed, cause a computing device to: define a plurality of steps of an operating room workflow for a medical procedure; link one or more instrument use events to at least a portion of the plurality of steps in the operating room workflow; trigger an instrument use event based an identification and classification of at least one object within a field of view of a real-time video feed in an operating room (OR); and automatically, in response to triggering the instrument use event, (1) advance the operating room workflow to a step linked to the instrument use event triggered by the instrument device recognition engine; and/or (2) perform a data collection event linked to the instrument use event triggered by the instrument device recognition engine.

Example 11 includes the subject matter of Example 10, and wherein there are further instruments to train a machine learning model that identifies and classifies at least one object within a field of view of a real-time video feed in an operating room (OR) with a plurality of photographs of objects to be detected.

Example 12 includes the subject matter of Examples 10-11, and wherein: the plurality of photographs of the objects to be detected includes a plurality of photographs for at least a portion of the objects that are rotated with respect to each other.

Example 13 includes the subject matter of Examples 10-12, and wherein: the at least one object is identified and classified within the field of view of the real-time video feed in the operating room (OR) based on a machine learning (ML) model.

Example 14 includes the subject matter of Examples 10-13, and wherein: a convolutional neural network (CNN) is to identify and classify at least one object within the field of view of the real-time video feed in the operating room (OR).

Example 15 includes the subject matter of Examples 10-14, and wherein: detecting of one or more objects within the field of view of the real-time video feed in the OR includes concurrent segmentation and localization.

Example 16 includes the subject matter of Examples 10-15, and wherein: detecting of one or more objects within the field of view of the real-time video feed in the OR includes occlusion reasoning.

Example 17 includes the subject matter of Examples 10-16, and wherein: triggering the instrument use event is based on detecting at least one object entering the field of view of the real-time video feed in an operating room (OR).

Example 18 includes the subject matter of Examples 10-17, and wherein: triggering the instrument use event is based on detecting at least one object leaving the field of view of the real-time video feed in an operating room (OR).

Example 19 includes the subject matter of Examples 10-18, and wherein: to determine the step linked to the instrument use event is determined as a function of a role-based setting.

Example 20 is a method for managing operating room workflow events. The method includes the step of receiving a real-time video feed of one or more of instrument trays and/or preparation stations in an operating room. One or more surgical instrument-use events are identified based on a machine learning model. The method also includes automatically advancing a surgical procedure workflow and/or triggering data collection events as a function of the one or more surgical instrument-use events identified by the machine learning model.

1. A computing device for managing operating room workflow events, the computing device comprising:
an instrument use event manager to: (i) define a plurality of steps of an operating room workflow for a medical procedure; and (ii) link one or more instrument use events to at least a portion of the plurality of steps in the operating room workflow;
an instrument device recognition engine to trigger an instrument use event based an identification and classification of at least one object within a field of view of a real-time video feed in an operating room (OR); and
a workflow advancement manager to, in response to the triggering of the instrument use event, automatically: (1) advance the operating room workflow to a step linked to the instrument use event triggered by the instrument device recognition engine; and/or (2) perform a data collection event linked to the instrument use event triggered by the instrument device recognition engine.

2. The computing device of claim 1, wherein the instrument device recognition engine is configured to identify and classify at least one object within the field of view of the real-time video feed in the operating room (OR) based on a machine learning (ML) model.

3. The computing device of claim 1, wherein the instrument device recognition engine includes a convolutional neural network (CNN) to identify and classify at least one object within the field of view of the real-time video feed in the operating room (OR).

4. The computing device of claim 1, wherein the instrument device recognition engine includes concurrent segmentation and localization for tracking of one or more objects within the field of view of the real-time video feed in the OR.

5. The computing device of claim 1, wherein the instrument device recognition engine includes occlusion reasoning for object detection within the field of view of the real-time video feed in the OR.

6. The computing device of claim 1, wherein the instrument device recognition engine is to trigger the instrument use event based on detecting at least one object entering the field of view of the real-time video feed in the operating room (OR).

7. The computing device of claim 1, wherein the instrument device recognition engine is to trigger the instrument use event based on detecting at least one object leaving the field of view of the real-time video feed in the operating room (OR).

8. The computing device of claim 1, wherein the workflow advancement manager is to determine the step linked to the instrument use event as a function of the identification and classification of the object detected by the instrument device recognition engine.

9. The computing device of claim 8, wherein the workflow advancement manager is to determine the step linked to the instrument use event as a function of a role-based setting.

10. One or more non-transitory, computer-readable storage media comprising a plurality of instructions stored thereon that, in response to being executed, cause a computing device to:
define a plurality of steps of an operating room workflow for a medical procedure;
link one or more instrument use events to at least a portion of the plurality of steps in the operating room workflow;
trigger an instrument use event based an identification and classification of at least one object within a field of view of a real-time video feed in an operating room (OR); and
automatically, in response to triggering the instrument use event, (1) advance the operating room workflow to a step linked to the instrument use event; and/or (2) perform a data collection event linked to the instrument use event.

11. The one or more non-transitory, computer-readable storage media of claim 10, further comprising instruments to train a machine learning model that identifies and classifies at least one object within the field of view of the real-time video feed in the operating room (OR) with a plurality of photographs of objects to be detected.

12. The one or more non-transitory, computer-readable storage media of claim 11, wherein the plurality of photographs of objects to be detected includes a plurality of photographs for at least a portion of the objects that are rotated with respect to each other.

13. The one or more non-transitory, computer-readable storage media of claim 10, wherein the at least one object is identified and classified within the field of view of the real-time video feed in the operating room (OR) based on a machine learning (ML) model.

14. The one or more non-transitory, computer-readable storage media of claim 10, wherein a convolutional neural network (CNN) is to identify and classify at least one object within the field of view of the real-time video feed in the operating room (OR).

15. The one or more non-transitory, computer-readable storage media of claim 10, wherein detecting of one or more objects within the field of view of the real-time video feed in the OR includes concurrent segmentation and localization.

16. The one or more non-transitory, computer-readable storage media of claim 10, wherein detecting of one or more objects within the field of view of the real-time video feed in the OR includes occlusion reasoning.

17. The one or more non-transitory, computer-readable storage media of claim 10, wherein triggering the instrument use event is based on detecting at least one object entering the field of view of the real-time video feed in the operating room (OR).

18. The one or more non-transitory, computer-readable storage media of claim 10, wherein triggering the instrument use event is based on detecting at least one object leaving the field of view of the real-time video feed in the operating room (OR).

19. The one or more non-transitory, computer-readable storage media of claim 10, wherein to determine the step linked to the instrument use event is determined as a function of a role-based setting.

20. A method for managing operating room workflow events, the method comprising:
receiving a real-time video feed of one or more of instrument trays and/or preparation stations in an operating room;
identifying one or more surgical instrument-use events based on a machine learning model; and
automatically advancing a surgical procedure workflow and/or triggering data collection events as a function of the one or more surgical instrument-use events identified by the machine learning model.
